(19) United States
(12) Patent Application Publication
(10) Pub. No.: US 2009/0187197 A1
(43) Pub. Date: Jul. 23, 2009

(54) Knit PTFE Articles and Mesh

(76) Inventors: Peter J. Roeber, Wallingford, PA (US); James W. Mann, Elkton, MD (US); Eileen M. Bailey, Landenberg, PA (US); Anand Narayan, Bear, DE (US)

Correspondence Address: Bridget C. Sciamanna;W. L. Gore & Associates, Inc., 551 Paper Mill Road, P.O. Box 9206, Newark, DE, 19714-9206, US

(21) Appl. No.: 12/184,544

(22) Filed: Aug. 1, 2008

Related U.S. Application Data
(63) Continuation-in-part of application No. 11/833,566, filed on Aug. 3, 2007.

(51) Int. Cl. A61B 17/08

(52) U.S. Cl. 606/151

(57) ABSTRACT

Disclosed is a knitted article, and a method of producing such an article, having at least one PTFE fiber with oriented fibrils forming multiple fiber cross-over points wherein PTFE fiber is self-bonded in at least one of the cross-over points.

### CROSS REFERENCE TO RELATED APPLICATIONS

This application is a continuation-in-part of co-pending application U.S. Ser. No. 11/833,566, filed Aug. 3, 2007.

### BACKGROUND OF THE INVENTION

Mesh fabric prostheses are used in various surgical procedures including repair of anatomical defects of abdominal wall, diaphragm, and chest wall, correction of defects in the genitourinary system, and repair of traumatically damaged organs such as the spleen, liver, or kidney. Hernia repairs are among the more common surgical operations that employ such prostheses.

Surgical repair mesh fabrics are constructed from a variety of synthetic fibers in the form of knitted and woven fabrics.

Ventral hernias can be repaired using open or laparoscopic techniques that include intraperitoneal or preperitoneal placement of a prosthetic biomaterial (e.g. mesh or patch). In the US it is estimated that about 16% (US Markets for Soft Tissue Repair 2006, Millennium Research Group) of ventral hernias are repaired using an open preperitoneal technique while in Europe the percentage is much higher, approximately 69% (European Markets for Soft Tissue Repair 2006, Millennium Research Group). In these cases, open knit meshes of polypropylene comprise the material of choice, providing high in-growth, the ability to treat if infected, and sufficient initial stiffness to enable ease of use during implantation of the prosthetic. However, it is reported in the literature that meshes constructed from polypropylene elicit a prolonged inflammatory response (Kiosterhalfen et al., Expert Rev Med Devices (2005); January 2(1): 103-17) (Klinge et al., Eur J Surg (1999); 165: 665-673) or chronic foreign body response (FBR). This tissue response may lead to potentially serious long-term complications such as mesh erosion, mesh migration, fistulas, aggressive adhesions when in contact with the viscera and a reduction in postoperative compliance leading to patient discomfort. Studies within the literature have evaluated methods to reduce these responses. The solution most commonly pursued is the use of monofilament, large pore, reduced material polypropylene meshes. These meshes present a reduced surface area for biological interaction thereby reducing the foreign body response. An improved solution is through substitution of polypropylene with a more biocompatible material such as polytetrafluoroethylene (PTFE). PTFE knit mesh currently exists in the market, for example one such brand is Bard® PTFE Mesh; however, it lacks the appropriate monofilament, reduced material, large pore structure. Even so, due to the inherently lower modulus of the material, PTFE knit articles have inferior handling (stiffness), compared to the preferred characteristics of polypropylene mesh. Conventional methods of raising the stiffness of the knit article result in increased foreign body and or the introduction of an additional material that takes away from the biocompatibility of the construct. The ideal PTFE knit prosthetic mesh should be constructed such that it combines the ideal structure and material without sacrificing the desired handling. The present invention addresses this limitation and enables the creation of a highly biocompatible, monofilament, reduced material, large pore prosthetic mesh with appropriate handling for the reconstruction of hernias and other soft tissue deficiencies.

### SUMMARY OF THE INVENTION

In one embodiment of the invention is provided a knitted article with a knitted structure having at least one PTFE fiber with oriented fibrils forming multiple fiber cross-over points wherein the PTFE fiber is self-bonded in at least one of the cross-over points. The fibrils in the knitted structure are able to self-bond while oriented essentially non-parallel to each other. In another embodiment the PTFE fiber is a monofilament PTFE fiber.

### DESCRIPTION OF DRAWINGS

FIGS. 1A-1B show an electron microscopy view which illustrates a PTFE knitted article before heat treatment.

FIGS. 1C-1D illustrate PTFE knitted articles after heat treatment.

FIG. 2 is a diagram of various knitted mesh examples of shapes and geometries for the PTFE knitted article.

FIGS. 3A-3E show a cross-section diagram of a PTFE knitted article used in combination with layered absorbable, non-absorbable, or biological cells.

FIG. 4—In the BB test a ball is pushed into the mesh and the displacement and load are recorded.

FIG. 5 describes BB measurements for three different knit samples as described in Example 5.

### DETAILED DESCRIPTION OF THE INVENTION

A knitted PTFE surgical mesh is provided which exhibits both a desirable material and unique handling features, without the need of a bonding additive. The unique ability of the PTFE knit to form articles without the addition of bonding additives, to achieve tissue integration, favorable anti-inflammation results, and good biomechanical resistance for soft tissue repair is an unexpected result derived from the present invention.

A knitted article is constructed of a yarn having at least one PTFE fiber. The term PTFE is meant to be inclusive of expanded polytetrafluorethylene (ePTFE). The PTFE fiber comprises oriented fibrils. The PTFE fiber may be microporous or non-microporous. In one aspect the PTFE fiber 4 is a monofilament PTFE fiber. In another aspect, the PTFE fiber may be at least two different PTFE fibers having differing deniers, density, lengths or dimensional differences. In another aspect of the present invention, a multiple strand yarn which is comprised of at least one PTFE fiber and at least one other type of fiber that is not PTFE may be knitted. In this aspect, the PTFE fiber in the multiple strand yarn self-bonds at the crossover points of PTFE fiber crossed over PTFE fiber. The PTFE fiber may be the same strand or a differing strand in the same multiple strand yarn. A self bonding at a cross-over point may be created without an adhesive using self-bonding techniques.

A non-microporous PTFE fiber prevents the penetration and harboring of bacteria. Open knitted structures provide for tissue in-growth and may provide for better infection treatable implants. A PTFE knit is a monofilament structure in its simplest form. The structure may be filled or loaded with therapeutic agents, to facilitate, for example, drug delivery, as desired.

The PTFE fiber is knitted into an article, forming multiple fiber cross-over points formed where the PTFE fiber is in contact with itself. The PTFE yarn is configured into the desired knit pattern and the knit pattern is then heat treated to increase stiffness. The heating bonds the PTFE fiber onto itself in at least one cross-over point.

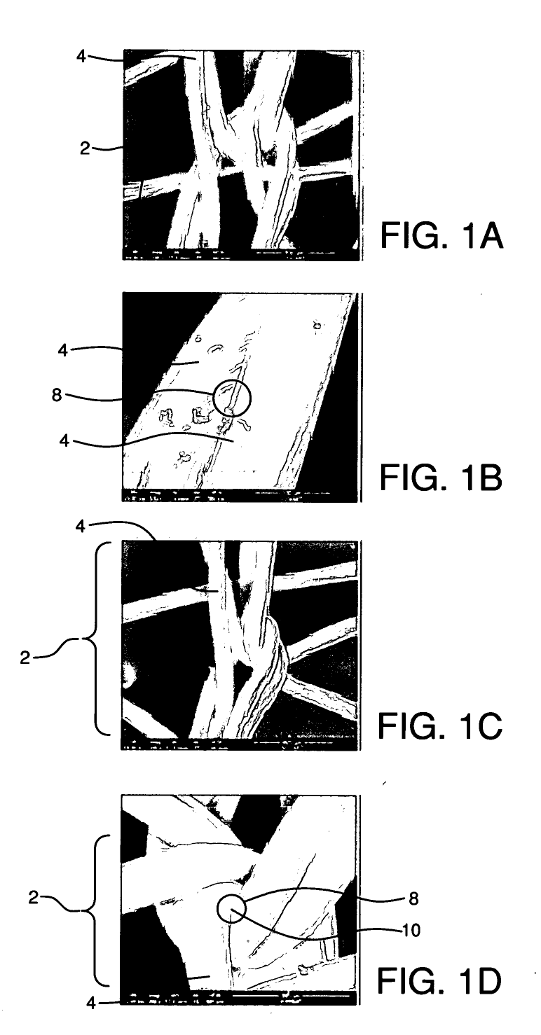

FIG. 1A shows an example of the structure of a PTFE knitted article prior to heat treatment. A PTFE fiber 4 is formed into a knitted structure 2 so that the PTFE fiber 4 forms multiple fiber crossover points 8 wherein the PTFE fiber 4 has a self-bonded region 10 in at least one of the cross-over points 8. FIG. 1B shows a crossover point 8 of two PTFE fibers 4 in a knitted structure wherein no self-bond regions occur as illustrated by no evidence of bonding between the areas of contact allowing the fibers to move independent of each other.

One way to form the knitted structure 2 of the present invention is by configuring the PTFE fiber 4 into a knit pattern formed onto or attached to a restraining means 12, and exposing the knitted structure 2 to heat while the knitted structure 2 remains fully constrained, thus preventing the knitted structure from contracting or moving. The restraining means 12 may be a pin hoop, clasp, frame, or any suitable mechanism which allows the knit to be firmly affixed and heated. When exposed to heat, the PTFE fiber 4 will shrink in a primarily longitudinal manner. This shrinkage causes the constrained article on the pin hoop to become taut which creates pressure at the cross-over points or the intersections of the fibers within the knitted article.

It was unexpected that PTFE fibers self-bond without externally applied pressure and heat or the use of a bonding agent. This type of bonding is an advantage in producing a fully PTFE or fully ePTFE knit article. The temperature at which the fibrils are able to self-bond ranges from between 327-400° C. A controlled temperature of between 350-370° C. and a controlled exposure time for a period of about 5-10 minutes may be desired in the processing of meshes.

The stiffness of the knit may be increased with longer durations of heating at a controlled temperature setting. The self-bonding of two or more PTFE fibers with oriented fibrils is a result of being contacted under heat and fiber to fiber tension, allowing the fibers to fuse or lock at the interfaces of the fibers without the need of a bonding agent. When the fibrils are fused together, they are prevented from relative movement with regard to each other, referred to as cross-fibril locking. The knitted structure 2 may be cooled prior to removing from the restraining means. The cooled article may then be cut into a finished geometry using various methods known in the art. The bonding at the multitude of fiber intersections within the textile results in a significant increase in the stiffness of the article providing more preferred handling characteristics. For instance, if desired, the resulting stiffness of a knit structure can be increased by at least 50%, over the same knit structure in a non-self-bonded form. Accordingly, various knit patterns and fiber deniers may result in differing stiffness measurements.

The stiffness of PTFE knitted structures may be modulated as desired by choice of material thickness and composition, and measured by standard tests, i.e. INDA Standard Test IST 90.3 (95) Handle-O-Meter of Nonwoven Fabrics. For instance, a PTFE knitted structure in the form of a single thickness mesh may be formed having a mass between 50-70 g/m², 70-90 g/m², or 90-165 g/m² and having a stiffness of greater than 25 g, 35 g, or 50 g, respectively.

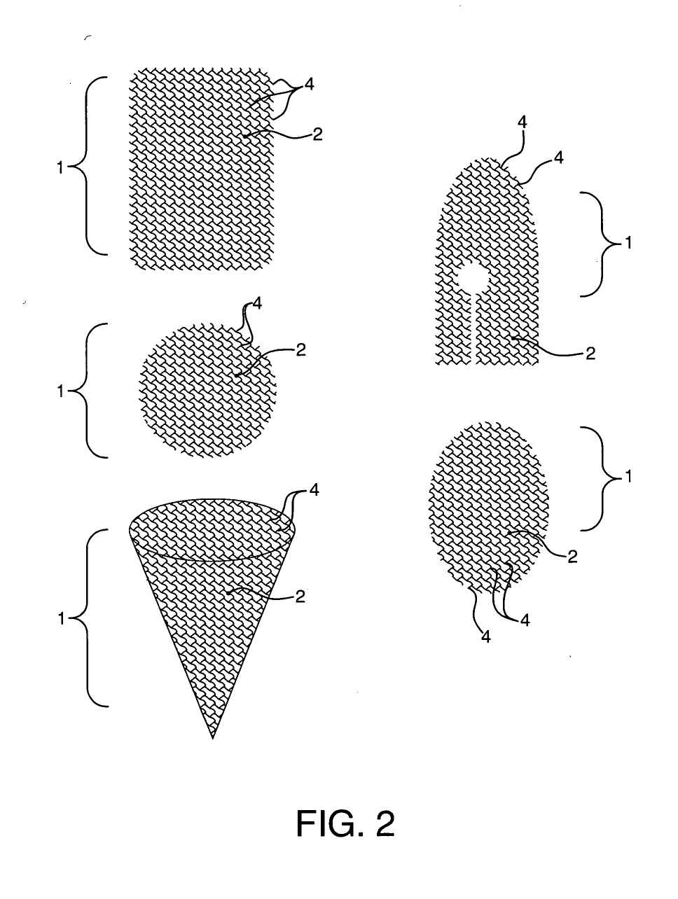

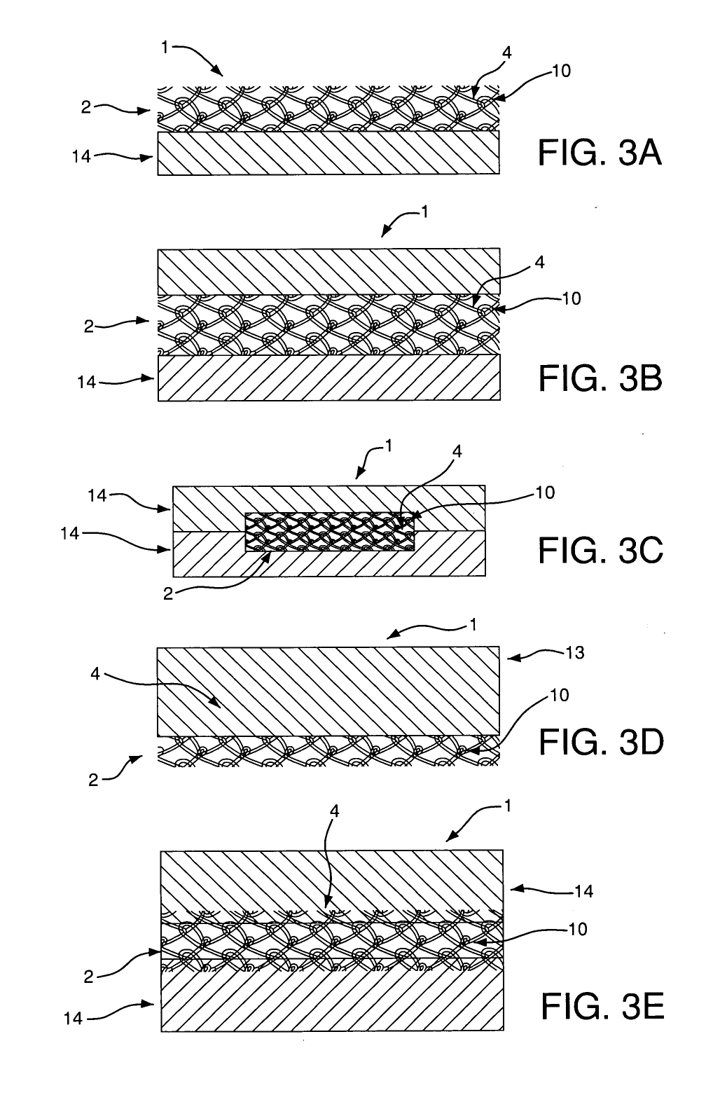

FIG. 2 shows diagrams of articles 1 having knitted structures 2 formed of PTFE fiber 4. The knitted structure in these embodiments is solely in the form of a knitted mesh of various shapes and geometry. As shown in FIGS. 3A-3E below, the knitted structure may be combined with other elements to form desired articles.

A surprising feature of the knitted structure 2 is that the PTFE fiber 4 comprises oriented fibrils 6 which may be self-bonded in a relatively non-parallel configuration which allows the structure to have increased pattern stability and stiffness.

A PTFE knit structure of the present invention is able to be easily handled due to the self-bonded regions, hence providing additional stability and stiffness to prevent twisting and wrinkling associated with traditional PTFE articles. Similarly, articles formed of the present invention may provide a patient with a more comfortable article which provides support to the injured area and also provides the patient with greater flexibility of the in situ article.

As exemplified in FIGS. 3A-3E, the PTFE knitted structure 2 may be fabricated into articles having at least one knitted structure and at least one other material to form a composite structure. FIG. 3A shows an article 1 formed of a PTFE knitted structure 2 and a functional layer 14. The knitted structure comprises at least one PTFE fiber 4 formed into the knitted structure 2 so that the PTFE fiber 4 forms multiple fiber cross-over points wherein the PTFE fiber 4 has a self-bonded region 10 in at least one of the cross-over points. The functional layer 14 may comprise either a non-absorbable, absorbable, or resorbable material. Examples of non-absorbable materials include but are not limited to ePTFE, polypropylene, polyester, and fluoropolymers. Examples of absorbable materials include but are not limited to polymers or co-polymers or blends of absorbable materials. The knitted structure 2 and a functional layer 14, as illustrated in FIGS. 3A-3C may be laminated or joined by various methods known in the art or as described in the examples below.

FIG. 3B shows an article having a composite structure formed of a PTFE knitted structure 2, and two functional layers 14. In this figure, one layer comprising a PTFE knitted structure 2 having a top side and a bottom side is joined to a functional layer 14 on its top side and a functional layer 14 on its bottom side forming an article. As discussed above, the functional layer 14 may be absorbable or non-absorbable or a combination of absorbable and non-absorbable material. It is also within the scope of the invention that more than one knitted structure may be present in a single article. Further, it is similarly within the scope of the invention that more than two functional layers may be present in a single article. The article may be used in various manners including as a surgical material, surgical mesh, hernia repair, soft tissue repair, soft tissue reinforcement, repair of anatomical defects, correction of defects in the genitourinary system, repair of traumatically damaged organs including spleen, liver, kidney, or any combination thereof, anatomical defect repair including defects in the abdominal wall, diaphragmatic, chest wall, or any suitable use or combination thereof.

FIG. 3C shows the PTFE knitted structure 2 fabricated into an article such that the knitted structure is fully enclosed between two functional layers 14 to form a composite structure. The functional layers may be comprised of similar or different materials, and may also each have similar or different properties, such as rate of absorption, drug eluting properties, or mechanical properties such as stiffness. The functional layers may be joined or bonded together as discussed above including via lamination, an absorbable adhesive, or other means of joining.

The PTFE knitted structure 2 can further be gravure printed with a discrete pattern of adhesive. The pattern can be designed such that it closely matches that of the knit pattern allowing maximum surface coverage with absorbable adhesive. The knitted structure 2, with adhesive present, can then be laminated to a non-woven self adhering web. Other methods for manufacturing the composite article include but are not limited to thermal bonding, sewing, solvent bonding, using a tie layer of a separate absorbable material to join layers or permanent layer coatings or similar known means for forming composite articles. One skilled in the art could use these processes to achieve a multi-layer composite article of at least one PTFE knitted structure 2 and at least one functional layer.

Other examples of articles of the present invention include PTFE knit structures formed into surgical meshes and incorporated into multi-layer composite constructions having individual absorbable and non-absorbable layers imbibed throughout at least a portion of the PTFE knitted structure 2, as illustrated in FIG. 3D or 3E.

FIG. 3D shows an article 1 comprising a PTFE knitted structure 2 joined with a biological material 13. The biological material is a modulating substance which either enhances wound healing, provides induced cellular or tissue growth of the host to which it contacts or provides induced cellular or tissue growth of the biological material itself.

FIG. 3E shows an article having a composite structure formed of a PTFE knitted structure 2, and two functional layers 14. In this figure, one layer comprising a PTFE knitted structure 2 with a self-bonded region 10, and having a top side and a bottom side is joined to a functional layer 14 on its top side and a functional layer 14 on its bottom side forming an article. The functional layer 14 may be imbibed into the knitted structure as shown. It is anticipated that the functional layers could be either partially or fully imbibed into the knitted structure. The functional layer 14 may be absorbable or non-absorbable or a combination of absorbable and non-absorbable material. It is also within the scope of the invention that more than one knitted structure may be present in a single article.

In one embodiment, the knitted article or mesh comprises a knitted structure having at least one PTFE fiber with oriented fibrils. The PTFE fiber forms multiple fiber cross-over points wherein said PTFE fiber is self-bonded (bonded onto itself at the cross-over point of the fiber to form a bond without the use of an adhesive) in at least one cross-over point. The PTFE fiber is a porous structure with an initial stiffness and a second stiffness. The second stiffness is less than the initial stiffness. The initial stiffness decreases to reach a second stiffness when the porous structure is subjected to surface tension. It is desirable that the knitted article soften or decrease stiffness under a tension force equal or less than the typical abdominal muscle tension exerted by a person when an individual is sitting, standing, coughing, or jumping. The softening or decrease of stiffness under a tensional force occurs when a bond in at least one of said cross-over points is broken when the knitted structure is subjected to a surface tension equal or less than intraabdominal pressure, as defined in examples below. The fiber may be knitted, woven or braided.

The PTFE surgical mesh may be coated with absorbable or drug-eluting compositions as described in the examples below or coating methods known in the art. Additionally, it is within scope of the present invention that a bioactive agent, antimicrobial agents, and/or antibiotics may be embedded in the drug-eluting compositions prior to coating the PTFE surgical mesh. The bioactive may be an analgesic, non-steroidal-anti-inflammatory drug (NSAID), or an anesthetic.

### EXAMPLES

### Example 1

Table 2 illustrates a typical example of knitted mesh as utilized by this invention. These examples are illustrative only and are not intended to limit the scope of the present invention. Various knit patterns may be used to form the PTFE knits used in the present invention.

TABLE 2

| Knit Pattern | | | | | | |
|---|---|---|---|---|---|---|
| Examples | A | B | C | D | E | F |
| Filament | Mono | Mono | Mono | Mono | Mono | Mono |
| Knit Type | Dual | Dual | Dual | Dual | Dual | Dual |
| | Bar | Bar | Bar | Bar | Bar | Bar |
| WPI [wales per | 19 | 19 | 19 | 18 | 16 | 19 |
| inch] | | | | | | |
| CPI [Courses | 24 | 38 | 16 | 21 | 23 | 29 |
| per Inch] | | | | | | |
| PTFE Fiber | 195 | 195 | 195 | 195 | 195 | 195 |
| [denier] | | | | | | |
| Mesh Weight | 63.4 | 89.5 | 123.3 | 135.5 | 163.8 | 164.4 |
| [g/m2] | | | | | | |
| Pore Size | 2600 | 1700 | 825 | 700 | 2100 | 425 |
| (approx.) [μm] | | | | | | |

### Example 2

### Stiffness Measurement

The overall hand, or average textile stiffness, was measured for each of the knit examples in Table 1 according to INDA Standard Test IST 90.3 (95) Handle-O-Meter Stiffness of Nonwoven Fabrics. A summary table of original stiffness can be found below in Table 3. As seen by Table 2 and Table 3, various knit patterns and fiber deniers may result in differing stiffness measurements.

### Example 3

### Stiffening Treatment

The untreated PTFE knits exemplified in Table 2 were treated to increase stiffness by attaching the textile to a pin hoop such that when exposed to heat the material will be fully constrained, thus preventing the article from contracting. The pin hoop is constructed of stainless steel, has a diameter of approximately 24 inches, and has 0.020 inch pin needles approximately 1 inch in length that are spaced evenly every 1-2 inches around the perimeter. The pin hoop, with textile firmly affixed, was then heated to 365° C. for 7 minutes. This treatment results in self-bonding at the fiber cross-over points.

### Example 4

The article described in example 3 was cooled to room temperature, removed from the pin hoop. The cooled article was cut into a finished geometry. The bonding at the multitude of fiber intersections (self-bonds) within the textile results in a significant increase in the stiffness of the article providing more preferred handling characteristics. Table 3 discloses the stiffness measurements of PTFE knits disclosed in Table 2 before and after self-bonding at the fiber cross-over points.

TABLE 3

| Knit Examples | A | B | C | D | E | F |
|---|---|---|---|---|---|---|
| Mesh Weight [g/m2] | 63 | 89 | 123 | 135 | 164 | 164 |
| Initial Stiffness (g) | 13 | 13 | 21 | 36 | 36 | 41 |
| Final Stiffness (g) | 28 | 46 | 180 | 155 | 415 | 308 |
| Percent Increase (%) | 221% | 362% | 860% | 430% | 1144% | 760% |

Although several examples are described here, one skilled in the art may easily influence mesh mass by changing stitch pattern, fiber denier, or fiber density to influence final mesh stiffness. One skilled in the art may also use different constraining and thermal treatment methods to obtain similar results.

### Example 5

### Stiffness Change Upon Deformation

A knit sample was made with the following characteristics:
Knit Pattern: Hexagonal
Knit Type Dual Bar
Filament: Mono
WPI: 28+/−5
CPI: 44+/−5
PTFE fiber diameter: <=127 um
Mesh Weight: 2.8+/−0.4 oz/yd²
Pore Size>1000 microns
Experimental Temperature Room Temperature

The above sample was heat treated at 365° C. for 3 minutes. Multiple samples, 4″×4″ in size, were cut from the above heat treated knit.

### Each Sample was Subjected to the Following:

The sample was loaded into the jaws of an Instron (Model # 5564) Universal Testing Machine, such that the direction of strain was the machine direction of the knit. The sample was displaced to a distance of 9 mm, at a rate of 80 mm/min. The load vs. displacement data was acquired. From this data the modulus of the knit along the machine direction was obtained, referred to as first modulus. The modulus is defined as the slope of the Load vs. Displacement Curve. The sample was then removed, and turned 900 such that the direction of strain was now the cross web direction of the knit. The sample was subjected to a displacement of 9 mm at 80 mm/min rate. Upon completion the sample was turned 900 so that the direction of the pull was once again along the machine direction. The load vs. displacement data was again acquired, using the same 80 mm/min rate and a maximum displacement of 9 mm. The modulus of the knit in the machine direction thus obtained for the second time is referred to as the second modulus.

Seventeen samples were measured. The second modulus was lower than the first modulus for each sample tested. The average reduction in the modulus was 47.8% with a minimum change of 26.1% and a maximum change of 64.5%. The self-bonded knit lost a significant part of its stiffness, as represented by the change in modulus, upon being stretched to a maximum strain of approximately 10%.

### Example 6

### Abdominal Mesh Tension Calculation

The values for Mean Intraabdominal Pressure (IAP) were extracted from Cobb et al. (see Cobb et al. “Normal Intraabdominal Pressure in Healthy Adults.” J Surg Res. 2005 December; 129(2):231-5. Epub 2005 September 2) and used as representative pressure measurements to calculate Abdominal Mesh Tension. The Abdominal Mesh Tension is considered to be the tension exerted on an implanted knitted mesh or knitted article when the implanted mesh or article is used to repair a defect in the abdominal wall of an individual. The measurement is representative of the tension applied to the knitted mesh or article when the individual is undergoing light activity such as sitting, standing, coughing, or jumping.

The method for calculating Abdominal Mesh Tension (see Table 4 below) is described by Klinge, et al. (see Klinge et al. “Modified mesh for hernia repair that is adapted to the physiology of the abdominal wall.” Eur J Surg. 1998 December; 164(12):951-60):

Mesh Tensionₕₒₒₚ N/cm=PD/2

Mesh Tension_{longitude} N/cm=PD/4
Where:
P=Pressure (N/cm)
D=Diameter (32 cm)

TABLE 4

| | | | Abdominal | Abdominal |
|---|---|---|---|---|
| | | | Mesh | Mesh |
| | | | Tension | Tension |
| | Mean IAP | | (longitude) | (hoop) |
| Activity | mmHg | N/cm² | N/cm | N/cm |
| Supine | 1.8 | 0.024 | 0.192 | 0.384 |
| Standing | 20.0 | 0.267 | 2.136 | 4.272 |
| Sitting | 19.6 | 0.231 | 1.848 | 3.696 |
| Stairs | 68.9 | 0.919 | 7.352 | 14.704 |
| Bending at | 14.4 | 0.192 | 1.536 | 3.072 |
| waist | | | | |
| Bending at | 20.6 | 0.275 | 2.2 | 4.4 |
| knees | | | | |
| Light activity | | | | |
| Min | 1.8 | 0.024 | 0.192 | 0.384 |
| Max | 68.9 | 0.919 | 7.352 | 14.70 |

### Example 7

### Abdominal Mesh Tension Test

Load vs. Displacement data was obtained from a modified ASTM D3787-01 Standard Test Method for Bursting Strength of Textiles—Constant-Rate-of-Traverse (CRT) Ball Burst Test. The ASTM test was modified by changing The-Constant-Rate-of-Transverse (CRT) from 300 mm/min to 50 mm/min and also changing the diameter of the steel ball used in the test from 25 mm to 38 mm (The modified test is referred to as “BB” hereinafter).

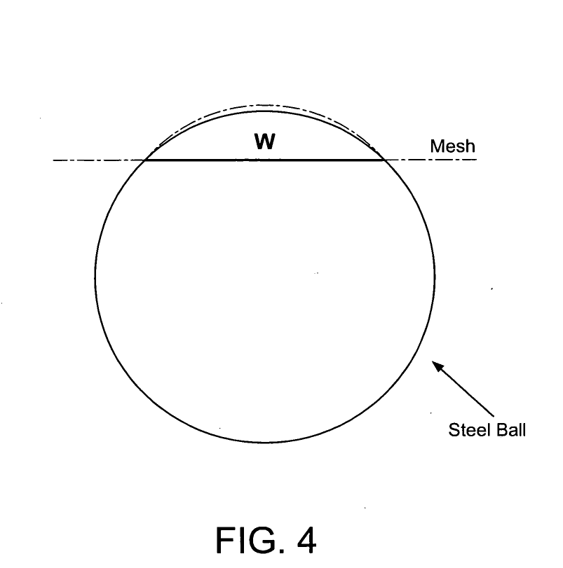

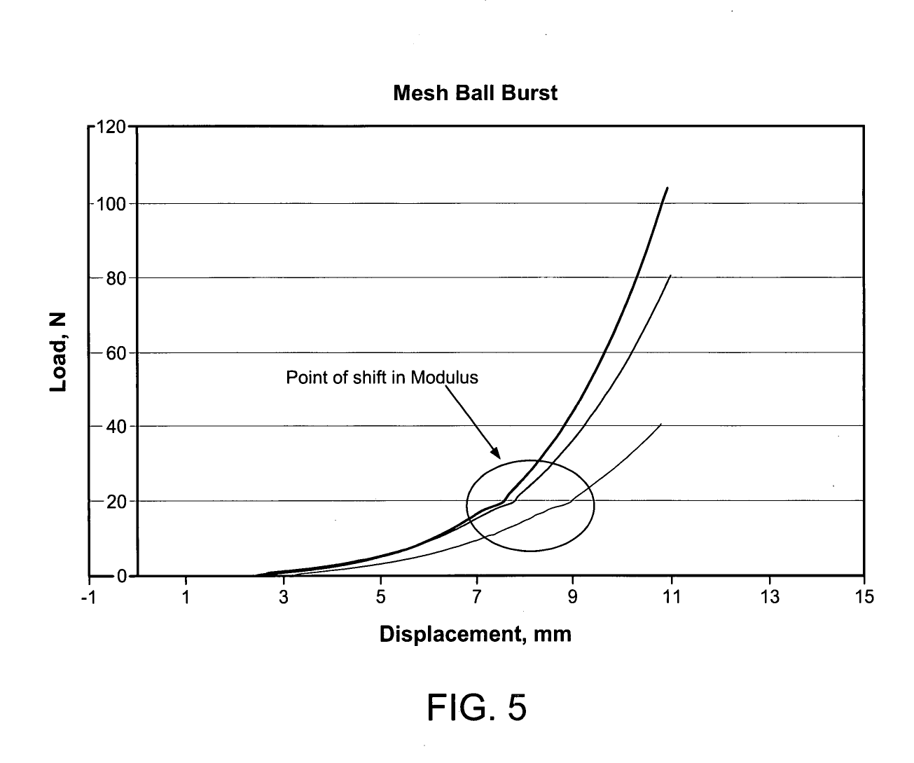

In the BB test a ball is pushed into the mesh and the displacement and load are recorded (see FIG. 4). FIG. 5 describes BB measurements for three different knit samples as described in Example 5. The experiment was conducted at Room Temperature. The Mesh Tension was calculated using the following formula:

Mesh Tension=Load/(π×W)

Wherein:

W: Width of Steel Ball at the point of displacement

Load: is Load at displacement

### Example 8

### Minimum Mesh Tension

The point of shift in modulus is defined as sudden decrease in the slope of the Load vs Displacement curve as depicted in FIG. 5. The surface tension of the mesh calculated at the point of shift in modulus is defined as Minimum Mesh Tension (hereinafter “MMT”). Minimum mesh tension is calculated as described in Example 7.

As depicted in FIG. 5 at point of shift in modulus the value of W is calculated to be 2.33 cm and the load is approximately 18N. Based on this information the average MMT for these samples is calculated to be 2.5 N/cm (MMT=18N/(3.14×2.33 cm)=2.5N/cm).

The invention claimed is:

1. A knitted article comprising:
a knitted structure having at least one PTFE fiber with oriented fibrils, said PTFE fiber forming multiple fiber cross-over points wherein said PTFE fiber is self-bonded in at least one of said cross-over points.

2. The knitted article of claim 1 wherein the fibrils are oriented non-parallel to each other.

3. The knitted article of claim 1 wherein said knitted article is a hernia repair mesh.

4. The knitted article of claim 1 wherein said knitted article is a soft tissue repair mesh.

5. The knitted article of claim 1 wherein said knitted article is a soft tissue reinforcement mesh.

6. The knitted article of claim 1 wherein said knitted article is for repair of anatomical defects.

7. The knitted article of claim 1 wherein said knitted article is for correction of defects in the genitourinary system.

8. The knitted article of claim 1 wherein said knitted article is for repair of organs.

9. The knitted article of claim 6 wherein said anatomical defect is abdominal wall.

10. The knitted article of claim 1 further comprising at least one functional layer.

11. A knitted article comprising:
a knitted structure having at least one monofilament PTFE fiber, said PTFE fiber forming multiple fiber cross-over points wherein said PTFE fiber is self-bonded in at least one of said cross-over points.

12. A knitted mesh comprising:
at least one PTFE fiber with oriented fibrils, said PTFE fiber being knitted into a mesh forming multiple fiber cross-over points wherein said PTFE fiber is self-bonded in at least one of said cross-over points, and wherein said mesh has a mass of between 50 and 70 g/m² and stiffness of greater than 25 g as determined by INDA Standard test Method 90.3.

13. A knitted mesh comprising:
at least one PTFE fiber with oriented fibrils, said PTFE fiber is knitted into a mesh forming multiple fiber cross-over points wherein said PTFE fiber is self-bonded in at least one of said cross-over points, and wherein said mesh has a mass of between 70 and 90 g/m² and stiffness of greater than 35 g as determined by INDA Standard test Method 90.3.

14. A knitted mesh comprising:
at least one PTFE fiber having oriented fibrils, said PTFE fiber is knitted into a mesh forming multiple fiber cross-over points wherein said PTFE fiber is self-bonded in at least one of said cross-over points, and wherein said mesh has a mass of between 90 and 165 g/m² and stiffness of greater than 50 g as determined by INDA Standard test Method 90.3.

15. A knitted article comprising:
a knitted structure having at least one PTFE fiber with oriented fibrils, said PTFE fiber forming multiple fiber cross-over points wherein said PTFE fiber is cross-fibril locked in at least one of said cross-over points.

16. A knitted article of claim 1 wherein said PTFE fiber is self-bonded in at least one of said cross-over points by heat treatment of said knitted article under tension.

17. A knitted article of claim 15 wherein said heat treatment is in the range of 350-375° C.

18. A mesh comprising:
at least one PTFE fiber with oriented fibrils, said PTFE fiber being knitted into a mesh forming multiple fiber cross-over points wherein said PTFE fiber is self-bonded in at least one of said cross-over points, and wherein said mesh has a mass of greater than 70 g/m² and stiffness of greater than 25 g as determined by INDA Standard test Method 90.3.

19. The knitted article of claim 1 wherein the knitted structure is solely PTFE.

20. The knitted article of claim 1 further comprising at least one additional non-knitted layer.

21. The knitted article of claim 1 further comprising at least one coating covering at least one portion of said knitted structure.

22. The article of claim 21 wherein the coating is a bioactive agent.

23. The article of claim 21 wherein the coating is an antimicrobial agent.

24. The article of claim 21 wherein the coating is an antibiotic.

25. A knitted article comprising a knitted structure having at least one PTFE fiber with oriented fibrils, said PTFE fiber forming multiple fiber cross-over points wherein said PTFE fiber is self-bonded in at least one of said cross-over points and wherein the knitted article has a porous structure with an initial stiffness and a second stiffness which is less than the initial stiffness when the porous structure is subjected to Abdominal Mesh Tension.

26. The knitted article of claim 25 wherein the second stiffness is at least 20% less than the initial stiffness.

27. The knitted article of claim 25 wherein the second stiffness is at least 30% less than the initial stiffness.

28. The knitted article of claim 25 wherein the second stiffness is at least 40% less than the initial stiffness.

29. The knitted article of claim 25 wherein the second stiffness is at least 50% less than the initial stiffness.

30. The knitted article of claim 25 wherein the second stiffness is at least 60% less than the initial stiffness.

31. The knitted article of claim 25 wherein the Abdominal Mesh Tension is less than 16 N/cm.

32. A surgical mesh comprising a knitted structure having at least one fiber forming multiple fiber cross-over points wherein said at least one fiber has a bond in at least one of said cross-over points and wherein said bond is broken when the knitted structure is subjected to a surface tension equal to or less than Intraabdominal Pressure.

33. A surgical mesh of claim 32 wherein the bond is a self-bond formed without adhesive.
